(19) United States
(12) Patent Application Publication
(10) Pub. No.: US 2007/0167669 A1
(43) Pub. Date: Jul. 19, 2007

(54) Ferromagnetic injection aneurysm repair

(75) Inventors: Nabil L. Muhanna, Gainesville, GA (US); David L. Schalliol, Oakwood, GA (US)

Correspondence Address: WOMBLE CARLYLE SANDRIDGE & RICE, PLLC, ATTN: PATENT DOCKETING 32ND FLOOR, P.O. BOX 7037, ATLANTA, GA, 30357-0037, US

(73) Assignee: Nabil L. Muhanna, M.D., Gainesville, GA (US)

(21) Appl. No.: 11/331,004

(22) Filed: Jan. 13, 2006

(51) Int. Cl. A61M 37/00; A61N 2/00; A61B 19/00; A61B 17/52

(52) U.S. Cl. 600/12; 600/9; 128/899

(57) ABSTRACT

A method of treating an aneurysm includes injecting a ferromagnetic material into the aneurysm and applying a magnetic field to the ferromagnetic material to form a ball in the aneurysm.

### FIELD OF THE INVENTION

One embodiment of the present invention is directed to a method for treating aneurysms. More particularly, one embodiment of the present invention is directed to a method of using ferromagnetic material to treat intracranial aneurysms.

### BACKGROUND INFORMATION

Saccular intracranial (cerebral) aneurysms occur when regions of weakness in the wall of intracranial arteries result in the development of balloon-like swellings on the sides of the arteries. They are important because they are prone to burst and cause hemorrhage over the surface of the brain (subarachnoid hemorrhage). This can result in death in over 30% of patients within 24 hours and a further 25-30% will die within the next four weeks without some form of surgical intervention.

Until recently the conventional treatment of cerebral aneurysms was to perform a neuro-surgical operation to place a clip across the neck of the aneurysm, analogous to tying the neck of a balloon. Over the past 15 to 20 years, endovascular techniques of aneurysm occlusion have evolved. The common principle to these techniques is that devices or materials are delivered by a tube (catheter) through the parent artery into the aneurysm where they induce clotting of the blood (thrombosis) in the aneurysm and effectively remove it from the circulation. The catheter is generally inserted via the femoral artery in the groin and the procedure monitored by x-ray fluoroscopy. The devices therefore have to be manipulated remotely, at a distance of approximately one meter without the help of direct vision. In the mid 1980s, balloons were used, made of latex or silicone rubber, to occlude the aneurysm. The results were poor and the mortality high. The reasons for this were: (1) It is rare that an aneurysm can be perfectly filled with one balloon; (2) If more than one balloon is used, large unfilled spaces must remain in the aneurysm (one sphere cannot be perfectly filled with more than one smaller spheres); and (3) Distension of the aneurysm will be the balloon(s) is likely to occur and cannot be seen or measured.

Prior art solutions having greater success include using soft coils of inert metal, generally platinum, to pack the aneurysm and induce thrombosis within it. The first coils were “free” in that they were simply pushed up the catheter into the aneurysm and once out of the catheter they could not be retrieved. This made the procedure difficult to control and hence risky.

In the 1990s a major advance in coil technology, the Guglielmi Detachable (“GD”) coil, was developed and it remains the only accepted device used in the endovascular treatment of cerebral aneurysms. Although essentially simply a soft platinum coil, it differs from the “free” coils in that it is captive and therefore controllable until the user chooses to release it.

The GD coil has proved very successful especially for aneurysms of the posterior intracranial circulation. It is often used in the treatment of aneurysms which are thought to be inoperable by other means. However, the GD coil has drawbacks, including relatively poor results in large (>1 cm diameter) or giant (>2 cm diameter) aneurysms, generally because of a tendency for the coil mass to pack down and for the neck region of the aneurysm to enlarge with time after treatment. Further problems include failure of endothelium to grow across the neck of treated aneurysms and a tendency for coils to prolapse out of wide necked aneurysms and obstruct the parent artery. In addition, early (procedural) or delayed (post treatment) rupture of the coils through the aneurysm wall.

Based on the foregoing, there is a need for an improved method for treating intracranial aneurysms.

### SUMMARY OF THE INVENTION

One embodiment of the present invention is a method of treating an aneurysm. The method includes injecting a ferromagnetic material into the aneurysm via a catheter or through normal blood flow and applying a magnetic field to the ferromagnetic material to form a ball in the aneurysm.

### BRIEF DESCRIPTION OF THE DRAWINGS

FIG. 1 is a cut-away view of a cranium that illustrates a method of treating an intracranial aneurysm in accordance with one embodiment of the present invention.

FIG. 2 is a cut-away view of the cranium that illustrates a method of treating an intracranial aneurysm in accordance with another embodiment of the present invention.

### DETAILED DESCRIPTION

One embodiment of the present invention is a method of treating an intracranial aneurysm by filling the aneurysm with ferromagnetic material or otherwise remotely maneuvering the material into a blood vessel to rest into the aneurysm sac. A magnetic field then causes the ferromagnetic material to stick together and form a ball that will permanently block the aneurysm.

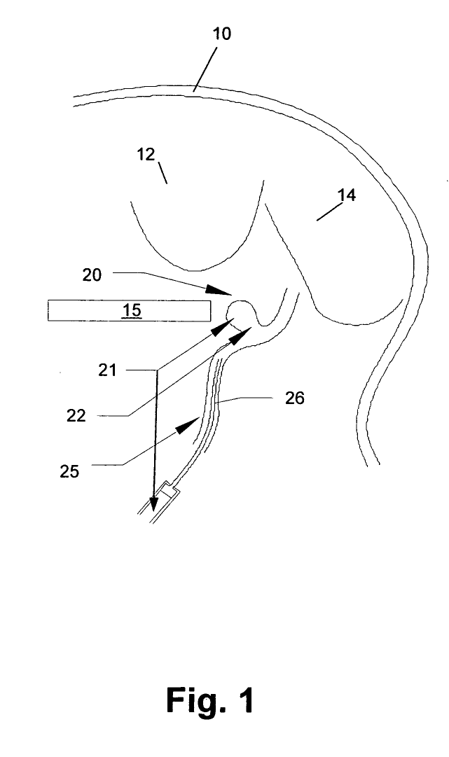

FIG. 1 is a cut-away view of a cranium 10 that illustrates a method of treating an intracranial aneurysm in accordance with one embodiment of the present invention. The method illustrated in FIG. 1 requires a craniotomy.

Cranium 10 encloses a temporal lobe 12 and frontal lobe 14. An aneurysm 20 of an artery 25 within cranium 10 has a mouth 22. In order to treat aneurysm 20, a catheter 26 filled with ferromagnetic material 21 is injected into artery 25. Catheter 26 fills up aneurysm 20 with the ferromagnetic material 21. In one embodiment, ferromagnetic material 21 comprises very fine iron particles of variable size. Using fluoroscopy, ferromagnetic material 21 is observed until aneurysm 20 is filled, at which time the flow of the material will be stopped. Ferromagnetic material 21 is injected in artery 25 in the proximity of the aneurysm. The value of the material 21 is measured to equal the value of the aneurysm, which may calculated by a computer.

After opening cranium 10, a magnet 15 is placed next to aneurysm 20. Magnet 15 causes the particles of ferromagnetic material 21 to be attracted to and accumulated within the aneurysm, and then by reversing the magnetic polarity, to stick with other and form a ball that will permanently block aneurysm 20. In one embodiment, the polarity of the magnetic field produced by magnet 15 is changed frequently from north to south from in order to accelerate magnetizing the particles of ferromagnetic material 21.

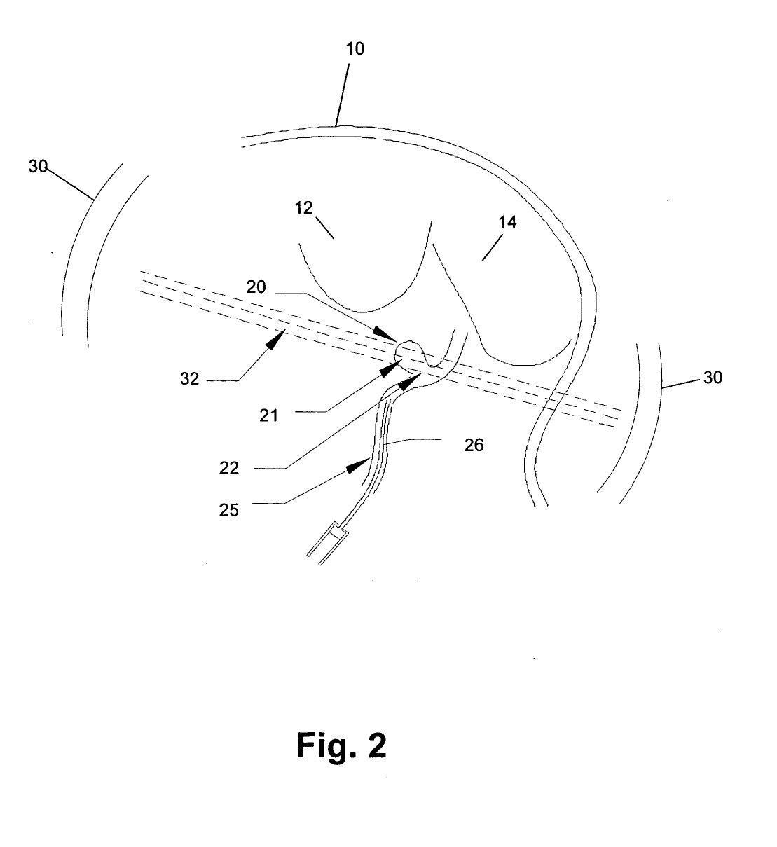

FIG. 2 is a cut-away view of cranium 10 that illustrates a method of treating an intracranial aneurysm in accordance with another embodiment of the present invention. The method illustrated in FIG. 2, as opposed to the method of FIG. 1, is non-invasive in that is does not require a craniotomy. Instead, after ferromagnetic material 21 is injected into aneurysm 20, a magnetic field 32 generated by magnets 30 from an magnetic resonance imaging (“MRI”) machine causes ferromagnetic material 21 to form a ball.

In one embodiment, magnetic field 32 is adjusted through MRI magnet 30 to shape the magnetic field as a line or pulling force. This pulling force is aligned with the aneurysm mouth 22, or the opening of the aneurysm to the blood stream. An angiogram of the cerebral aneurysm will show the mouth of the aneurysm with the magnetic line. The magnetic line or patient's head position is adjusted until the line passes through the mouth of the aneurysm. The patient is then positioned in the magnetic field with the head fixed with pins to prevent movement. The volume of ferromagnetic material that is injected is calculated in one embodiment via a computer, and is approximately equal to the internal volume of the aneurysm.

Several embodiments of the present invention are specifically illustrated and/or described herein. However, it will be appreciated that modifications and variations of the present invention are covered by the above teachings and within the purview of the appended claims without departing from the spirit and intended scope of the invention. For example, although embodiments disclosed are used to treat an aneurysm, the same methods can be used to treat a dilated blood vessel, or any tubular structure within a body.

What is claimed is:

1. A method of treating an aneurysm comprising:
injecting a ferromagnetic material into the aneurysm; and
applying a magnetic field to the ferromagnetic material.

2. The method of claim 1, said applying the magnetic field comprising:
performing a craniotomy; and
placing a magnet next to the aneurysm.

3. The method of claim 1, said applying the magnetic field comprising:
generating the magnetic field from a magnetic resonance imaging machine.

4. The method of claim 1, further comprising changing a polarity of the magnetic field when applying the magnetic field.

5. The method of claim 1, wherein said ferromagnetic material comprises a plurality of iron particles.

6. The method of claim 1, wherein injecting the ferromagnetic material comprises inserting a catheter in an artery.

7. The method of claim 1, wherein said aneurysm is an intracranial aneurysm.

8. A method of treating an intracranial aneurysm in an artery comprising:
inserting a catheter into the artery;
injecting a ferromagnetic material into the aneurysm through the catheter; and
applying a magnetic field to the ferromagnetic material to form a ball within the aneurysm.

9. The method of claim 8, said applying the magnetic field comprising:
performing a craniotomy; and
placing a magnet next to the aneurysm.

10. The method of claim 8, said applying the magnetic field comprising:
generating the magnetic field from a magnetic resonance imaging machine.

11. The method of claim 8, further comprising changing a polarity of the magnetic field when applying the magnetic field.

12. The method of claim 8, wherein said ferromagnetic material comprises a plurality of iron particles.

13. A method of treating a tubular structure within a body comprising:
injecting a ferromagnetic material into the tubular structure; and
applying a magnetic field to the ferromagnetic material.

14. The method of claim 13, said applying the magnetic field comprising:
generating the magnetic field from a magnetic resonance imaging machine.

15. The method of claim 14, further comprising changing a polarity of the magnetic field when applying the magnetic field.

16. The method of claim 14, wherein said ferromagnetic material comprises a plurality of iron particles.

17. The method of claim 14, wherein the tubular structure is an aneurysm.

18. The method of claim 14, wherein the tubular structure is a dilated blood vessel.
